(19) United States
(12) Patent Application Publication
(10) Pub. No.: US 2006/0239943 A1
(43) Pub. Date: Oct. 26, 2006

(54) Formula and method for providing protection from dermatitis, sunlight and/or insects

(76) Inventor: Nestor Sebastian Tomasi, Joliet, IL (US)

Correspondence Address: PATENTS+TMS, P.C., 2849 W. ARMITAGE AVE., CHICAGO, IL, 60647, US

(21) Appl. No.: 11/111,647

(22) Filed: Apr. 21, 2005

(51) Int. Cl. A61K 8/37; A61K 8/27; A61K 8/73

(52) U.S. Cl. 424/59; 424/70.13

(57) ABSTRACT

A formula and a method for providing protection of skin of a user from dermatitis, sunlight and/or an insect. The formula is applied to an area of the skin of the user prior to exposing the area to the dermatitis, the sunlight and/or the insect. The formula has one or more UV absorbers and/or a UV blocker to prevent effects of the sunlight and/or UV light on the area of the skin of the user. The UV blocker protects the area of the skin of the user from the dermatitis and/or the insect. The formula has one or more moisturizers, one or more waxes and/or an absorbant therein for softening the skin of the user.

### BACKGROUND OF THE INVENTION

The present invention generally relates to a formula and a method for providing protection from dermatitis, sunlight and/or insects. More specifically, the present invention relates to a formula and a method for providing protection from irritants on and/or degradation of an area of skin of a user. The area of the skin of the user may be affected with, for example, dermatitis, sunlight, bites from an insect and/or the like. The formula may have an ultraviolet light (hereinafter “UV”) blocker, one or more UV absorbers, one or more moisturizers, one or more waxes and/or an absorbant which may protect the area of the skin of the user. The formula may be applied to the area of the skin of the user prior to an exposure to the dermatitis, the sunlight and/or the insects. One or more UV absorbers may protect the skin of the user from harmful UV rays, such as, for example, UV-A rays, UV-B rays and/or UV-C rays. The formula may be applied to the area of skin to protect against a dermatitis, a chemical irritant, a microbiological irritant and/or a bite from an insect. The formula may prevent the area of the skin from being affected with the chemical irritant, the microbiological irritant and/or the dermatitis, such as, for example, urushiol induced contact dermatitis.

It is generally known that skin of a user may be affected by dermatitis, sunlight and insects. The dermatitis may be transferred from a plant to an area of a skin of a user. The dermatitis may be caused from, for example, poison ivy, poison oak, poison sumac and/or the like. The plant may have an active toxin therein which may cause the dermatitis to develop on the skin of the user. The active toxin of the plant may be transferred to the area of the skin of the user during contact of the area of the skin with, for example, the plant. The active toxin of the plant may be, for example, urushiol which may be an organic molecule. The active toxin, the organic molecule and/or the urushiol may bond to the area of the skin and/or may permeate tissue under the area of the skin of the user. As a result, the area of the skin and/or the tissue under the area of the skin may be an affected area of the skin of the user. Moreover, the active toxin on the affected area of the skin, the organic molecule and/or the urushiol may not be soluble and/or may not be removed with a solvent, such as, for example, water.

Traditionally, an organic solvent, such as, for example, a laundry soap may be applied to the area of the skin of the user to treat the dermatitis. The organic solvent may not reduce irritation of the affected area of skin caused by the active toxin, the organic molecule and/or the urushiol. Further, the organic solvent may spread the active toxin, the organic molecule and/or the urushiol to other areas of the skin of the user. Further, the organic solvent may not permeate the skin of the affected area and/or the tissue under the affected area of the user as easily as the urushiol may permeate the skin and/or the tissue of the user. Moreover, the organic solvent may not withdraw the urushiol from the tissue under the affected area of the skin of the user. Furthermore, the organic solvent may not prevent the urushiol and/or the organic molecule from bonding to the area of the skin of the user. As a result, the organic solvent may not protect the skin of the user from the urushiol and/or the organic molecule.

It is also generally known that UV light, such as, for example, sunlight may negatively affect the skin of the user. The UV light may include UV-A rays, UV-B rays, and/or UV-C rays. The UV light damages nucleic acids, such as, for example, deoxyribonucleic acid (hereinafter “DNA”) and/or ribonucleic acid (hereinafter “RNA”) of the user. The UV light may also break down folic acid in the user. The UV light may damage the skin of the user by, for example, tanning, burning and/or skin cancer, such as, for example, squamous cell carcinoma, basal cell carcinoma and/or malignant melanoma.

The UV light may cause an epidermis of the skin of the user to thicken to block the UV light. The UV light may cause melanocytes of the user to make increased amounts of melanin. As a result, the skin may darken and/or may result in a tan. The melanin may absorb the energy of UV light and may prevent the UV light from penetrating deeper into the tissues. Further, the UV light may cause premature aging of the skin of the user. Still further, the UV light may cause wrinkles in, irregular pigmentation of and/or leathering of the skin of the user.

The area of the skin of the user which is affected from the UV light may attract an insect thereon. The insect, such as, for example, a mosquito may bite into the skin of the user at the area affected by the UV light. Therefore, the area of the skin affected by the UV light may also be irritated by one or more bites from one or more insects. As a result, the user may experience discomfort and/or pain caused by the negative effects of the UV light and/or the one or more bites from one or more insects.

Traditionally, a product, such as, for example, moisturizing creams and/or makeup is used to treat an area of the skin which is negatively affected and/or may be affected by the UV light. Additionally, a treatment, such as, for example, chemical peels, alpha-hydroxy acids, tretinoin cream and/or laser skin resurfacing is used to improve an appearance of the area of the skin which is negatively affected by the UV light. However, significant damage to the area of the skin from the UV light is unlikely to be reversed by the product and/or the treatment. Furthermore, the product and/or the treatment does not prevent the UV light from negatively affecting the area of the skin of the user. Moreover, the product and/or the treatment does not prevent and/or protect against the dermatitis and/or one or more bites from one or more insects.

A need, therefore, exists for a formula and a method for providing protection from dermatitis, sunlight and/or insects. Additionally, a need exists for a formula and a method for providing protection from dermatitis, sunlight and/or insects which may be applied to an area of the skin of the user prior to the area being affected by the dermatitis, the sunlight and/or the insects. Further, a need exists for a formula and a method for providing protection from dermatitis, sunlight and/or insects which may have one or more UV blockers and/or UV absorbers to prevent the UV light from affecting the area of the skin. Still further, a need exists for a formula and a method for providing protection from dermatitis, sunlight and/or insects which may prevent effects of dermatitis, urushiol, a chemical irritant, a microbiological irritant and/or one or more bites from one or more insects on the skin of the user. Moreover, a need exists for a formula and a method for providing protection from dermatitis, sunlight and/or insects which prevents the urushiol, the chemical irritants and/or the microbiological irritants from the affected area of the skin of the user.

### SUMMARY OF THE INVENTION

The present invention generally relates to a formula and a method for providing protection from dermatitis, sunlight and/or insects. The formula may be applied to an area of skin of a user to prevent the dermatitis, the sunlight and/or the insect from affecting the area of the skin. The formula may have one or more UV absorbers and/or a UV blocker to prevent effects of the sunlight on the area of the skin. Additionally, the UV blocker may protect the area of the skin from dermatitis and/or may prevent an insect from biting the area of the skin. Further, the formula may have one or more moisturizers, one or more waxes and/or an absorbant therein for treating and/or for softening the skin of the user.

To this end, in an embodiment of the present invention, a formula for providing protection of an area of skin of a user from dermatitis or an insect wherein the formula is applied to the area of the skin of the user is provided. The formula has an ultraviolet light blocker wherein the ultraviolet light blocker prevents the area of the skin from being affected by dermatitis or an insect. Further, the formula has a first ultraviolet light absorber and a second ultraviolet light absorber. Moreover, the formula has a plurality of moisturizing agents and an absorbant.

In an embodiment, the ultraviolet light blocker is zinc oxide.

In an embodiment, the first ultraviolet light absorber is ethylhexyl methoxycinnamate or benzophenone-3

In an embodiment, the second ultraviolet light absorber is ethylhexyl methoxycinnamate or benzophenone-3

In an embodiment, the formula has a wax.

In an embodiment, the absorbant is corn starch.

In an embodiment, the formula has an anti-microbial agent.

In an embodiment, the formula has an anti-fungal agent.

In another embodiment of the present invention, a formula for providing protection of an area of skin of a user from dermatitis or an insect wherein the formula is applied to the area of the skin of the user is provided. The formula has an ultraviolet light blocker to protect the area of the skin from effects of the dermatitis and the insect. Further, the formula has a first ultraviolet light absorber to prevent the sunlight from affecting the skin of the user and petrolatum.

In an embodiment, the ultraviolet light blocker is zinc oxide.

In an embodiment, the first ultraviolet light absorber is ethylhexyl methoxycinnamate or benzophenone-3.

In an embodiment, the formula has an anti-microbial agent.

In an embodiment, the formula has a second ultraviolet light absorber.

In another embodiment of the present invention, a method for providing protection of an area of skin of a user from dermatitis or an insect is provided. The method has the step of providing a formula to block the dermatitis or the insect from affecting the area of the skin of the user wherein the formula has a zinc oxide, a first ultraviolet absorber, a petrolatum and an anti-microbial agent therein and further wherein the first ultraviolet absorber is ethylhexyl methoxycinnamate or benzophenone-3. Further, the method has the step of applying the formula to the area of the skin prior to exposing the area of the skin to the dermatitis or the insects.

In an embodiment, the method has the step of adding a moisturizing agent to the formula.

In an embodiment, the method has the step of adding a second ultraviolet absorber to the formula.

In an embodiment, the method has the step of adding an absorbant to the formula.

In an embodiment, the method has the step of adding a wax to the formula.

In an embodiment, the method has the step of homogenizing the formula.

In an embodiment, the anti-microbial agent is methylparaben or propylparaben.

It is, therefore, an advantage of the present invention to provide a formula and a method for providing protection from dermatitis, sunlight and/or insects.

Another advantage of the present invention is to provide a formula and a method for providing protection from dermatitis, sunlight and/or insects which may be applied to an area affected by the dermatitis, the sunlight and/or one or more bites from one or more insects.

And, another advantage of the present invention is to provide a formula and a method for providing protection from dermatitis, sunlight and/or insects which may have a UV blocker and/or one or more UV absorbers for applying to an area of skin of a user to protect the area from UV light.

Yet another advantage of the present invention is to provide a formula and a method for providing protection from dermatitis, sunlight and/or insects which may have UV blocker for protecting the skin from dermatitis and/or urushiol.

A further advantage of the present invention is to provide a formula and a method for providing protection from dermatitis, sunlight and/or insects which may have a UV blocker for preventing one or more insects from biting an area of skin of the user.

Moreover, an advantage of the present invention is to provide a formula and a method for providing protection from dermatitis, sunlight and/or insects which may have zinc oxide to prevent effects of dermatitis and/or sunlight at an area of skin of the user.

And, another advantage of the present invention is to provide a formula and a method for providing protection from dermatitis, sunlight and/or insects which may prevent UV-A rays, UV-B rays and/or UV-C rays from affecting an area of a skin of a user.

Yet another advantage of the present invention is to provide a formula and a method for providing protection from dermatitis, sunlight and/or insects which may have an absorbant and/or one or more moisturizers to prevent effects of dermatitis and/or sunlight on and/or to moisturize the skin of a user.

Another advantage of the present invention is to provide a formula and a method for providing protection from dermatitis, sunlight and/or insects which may have an anti-microbial agent and/or anti-fungal agent therein.

A further advantage of the present invention is to provide a formula and a method for providing protection from dermatitis, sunlight and/or insects which may have one or more waxes therein.

Moreover, an advantage of the present invention is to provide a formula and a method for providing protection from dermatitis, sunlight and/or insects which may be used for cosmetic purposes.

And, another advantage of the present invention is to provide a formula and a method for providing protection from dermatitis, sunlight and/or insects which may have propylparaben and/or methylparaben therein to preserve the formula and/or the system.

Yet another advantage of the present invention is to provide a formula and a method for providing protection from dermatitis, sunlight and/or insects which may have ethylexyl methoxycinnamate and/or benzophenone-3 to absorb UV light.

Additional features and advantages of the present invention are described in, and will be apparent from, the detailed description of the presently preferred embodiments and from the drawings.

### BRIEF DESCRIPTION OF THE DRAWINGS

FIG. 1 illustrates a flowchart for a method of compounding of the formula in an embodiment of the present invention.

### DETAILED DESCRIPTION OF THE PRESENTLY PREFERRED EMBODIMENTS

The present invention generally relates to a formula and a method for providing protection from dermatitis, sunlight and/or insects. The formula and the method may prevent chemical irritants and/or micro-biological irritants from affecting an area of a skin of a user. The formula may be applied to the area of the skin of the user prior to exposing the area to the dermatitis, the sunlight and/or the insects. The area of the skin of the user which may receive the formula may be, for example, a forearm, an arm, a face, a hand, a lip, a leg, a back, a foot and/or the like. The formula may be compounded into a form, such as, for example, a cream, an aqueous gel, a spray, a lotion, a lip stick, a powder, a complexion foundation and/or the like. The present invention should not be deemed as limited to the embodiments of a specific area of the skin of the user and/or a specific form of the formula.

The chemical irritants may be derived from an environment, a bite from an insect and/or from contact with, for example, plants, animals, cosmetics, clothes and/or the like. Further, the chemical irritants may be derived from an acute use, a chronic use or an exposure basis. The chemical irritants may cause a dermatitis, such as, for example, urushiol induced contact dermatitis. The dermatitis may be caused from a plant of the ginkgoaceae family or the anacardiaceae family, such as, for example, poison ivy, poison oak, poison sumac and/or the like. The present invention should not be deemed as limited to the embodiments of a specific dermatitis caused by the chemical irritants and/or a specific plant and/or a specific plant family causing the dermatitis.

The plant may have an active toxin thereon which may cause the dermatitis to develop on the skin of the user. The active toxin of the plant may be, for example, urushiol and/or the like. The formula may block the active toxin from contacting the area of the skin of the user. As a result, the formula may prevent the active toxin of the plant from being transferred to an area of the skin of the user during contact between the area of the skin with the plant. The formula may prevent the active toxin and/or the urushiol from bonding to the area of the skin and/or from permeating tissue under the area of the skin of the user. As a result, the area of the skin and/or the tissue under the area of the skin may be protected from the active toxin and the urushiol by the formula. The present invention should not be deemed as limited to the embodiments of a specific active toxin of the plant.

The formula may have, for example, an ultraviolet light (UV) blocker therein to prevent a UV light, such as, for example, UV-A rays, UV-B rays and/or UV-C rays from damaging and/or affecting the area of the skin of the user. The UV blocker may be, for example, zinc oxide. The UV blocker may have a weight percentage range of 0.5% to 4% of a total composition of the formula. The UV blocker may protect the skin of the user from and/or may block the UV light and/or the sunlight. The UV blocker may prevent the active toxin, the chemical irritants and/or the urushiol from contacting and/or bonding to the area of the skin of the user. Moreover, the UV blocker may discourage and/or may prevent one or more insects from biting the area of the skin of the user. The present invention should not be deemed as limited to the embodiments of a specific UV blocker in the formula.

The formula may have one or more UV absorbers therein for absorbing the UV light. One of the UV absorbers may be, for example, ethylexyl methoxycinnamate, benzophenone-3 or the like. The UV absorbers may have a weight percentage range of 8.5% to 11% of a total composition of the formula. One or more of the UV absorbers may protect the skin of the user from and/or may absorb the UV light and/or the sunlight. The present invention should not be deemed as limited to the embodiments of a specific UV absorber in the formula.

The formula may have one or more moisturizers, one or more waxes and/or an absorbant therein to moisturize, to treat, to condition and/or to soften the skin of the user. One of the moisturizers may be, for example, petrolatum, mineral oil, ethylhexyl palmitate and/or the like. The moisturizers may have a weight percentage range of 30% to 37% of a total composition of the formula. One of the waxes may be lanolin, paraffin, beeswax and/or the like. One or more of the waxes may be, for example, a hydrocarbon, an antioxidant or an anti-inflammatory. The waxes may have a weight percentage range of 20% to 25% of a total composition of the formula. The absorbant may be, for example, Zea Mays starch, corn starch and/or the like. The starch may have a weight percentage range of 0.5% to 5% of the total composition of the formula. The present invention should not be deemed as limited to the embodiments of a specific moisturizer, a specific wax and/or a specific absorbant in the formula.

The formula may have one or more anti-microbial agents therein. One or more anti-microbial agents may be water soluble and/or an anti-fungal agent. One of the anti-microbial agents may be, for example, propylene glycol, diazolidinyl urea, methylparaben, propylparaben and/or the like. The anti-microbial agents may enhance the co-solvency of the formula. The anti-microbial agents may have a weight percentage range of 0.20% to 0.50% of the total composition of the formula. The present invention should not be deemed as limited to the embodiments of a specific anti-microbial agent in the formula.

The formula may have sodium borate and/or a solvent, such as, for example, water therein. The water may have a weight percentage range of 30% to 34% of the total composition of the formula. The sodium borate may have a weight percentage range of 0.2% to 1.8% of the total composition of the formula. The present invention should not be deemed as limited to the embodiments of a specific solvent in the formula.

A first treatment of the formula may be applied to the area of the skin of the user prior to the area being exposed to the dermatitis, the sunlight and/or the insects. The formula may be rubbed onto the skin at the area of the skin of the user. The area of the skin of the user may absorb an absorbed portion of the formula. An unabsorbed portion of the formula may not be absorbed by the skin of the user. The unabsorbed portion of the formula may coat and/or may cover the area of the skin of the user.

The unabsorbed portion of the formula may prevent the dermatitis, the active toxins, the urushiol, the chemical irritants and/or the microbiological irritants from contacting and/or from bonding to the area of the skin of the user. Further, the unabsorbed portion of the formula may block and/or may absorb the UV light and/or the sunlight. Moreover, the unabsorbed portion of the formula may discourage and/or may prevent one or more insects from biting the area of the skin of the user. As a result, the unabsorbed portion of the formula may protect the area of the skin of the user from the dermatitis, the active toxins, the urushiol, the chemical irritants and/or the microbiological irritants, the UV light, the sunlight and/or one or more insects.

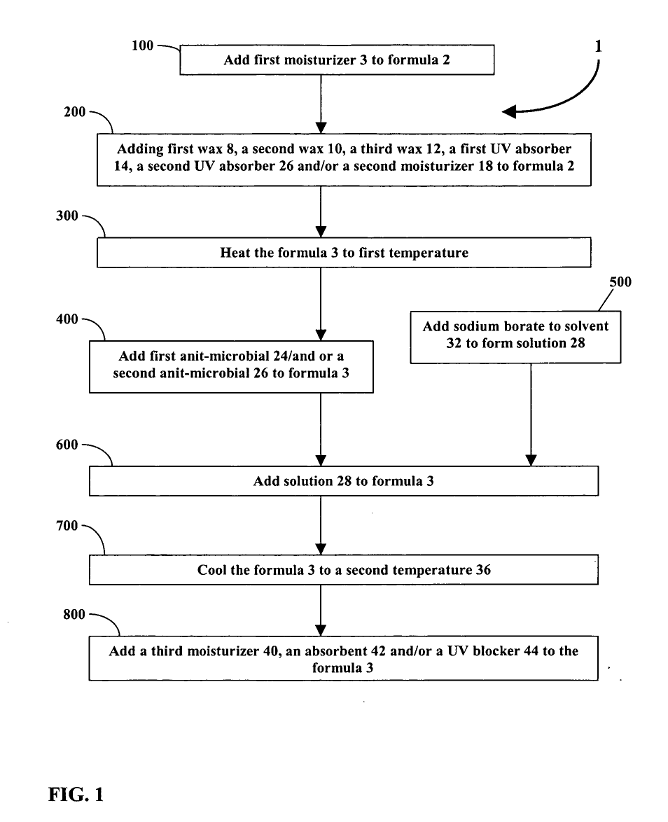

FIG. 1 illustrates a flowchart of a method 1 for compounding of the formula 3 in an embodiment of the present invention. The compounding of the formula 3 may occur in a container, such as, for example, a stainless steel tank. A first moisturizer 2 may be added to the container, may be mixed and/or may be heated in the container as illustrated at step 100. The first moisturizer 2 may be, for example, petrolatum. In step 100, the first moisturizer 2 may be heated between seventy-five (75) degrees Celsius and eighty (80) degrees Celsius. The present invention should not be deemed as limited to the embodiments of a specific container used during the compounding of the formula 3.

At step 200, a first wax 8, a second wax 10, a third wax 12, a first UV absorber 14, a second UV absorber 16 and/or a second moisturizer 18 may be added to the first moisturizer 2 to compounding the formula 3. The first wax 8, the second wax 10, the third wax 12, the first UV absorber 14, the second UV absorber 16 and/or the second moisturizer 18 may be mixed with and/or heated with the first moisturizer 2. The first wax 8 may be, for example, lanolin. The second wax 10 may be, for example, paraffin. The third wax 12 may be, for example, beeswax. The first UV absorber 14 or the second UV absorber 16 may be, for example, ethylhexyl methoxycinnamate and/or benzophenone-3. The second moisturizer 18 may be, for example, ethylhexyl palmitate.

The formula 3 may be stabilized at and/or heated to a first temperature as illustrated in FIG. 1 at step 300. The first temperature may be, for example, seventy-five (75) degrees Celsius. A first anti-microbial 24 and/or a second anti-microbial 26 may be added to the formula 3 as illustrated at step 400. The first anti-microbial 24 or the second anti-microbial 26 may be, for example, methylparaben or propylparaben. The formula 3 may be mixed into solution.

A solution 28 may be formed at step 500. The solution 28 may have sodium borate which may be added to a solvent 32, such as, for example, water. The solution 28 may be agitated into solution and/or may be heated to the first temperature 500. As illustrated in FIG. at step 600, the solution 28 may be added to the formula 3 and/or may be mixed into the formula 3 for a duration of time between, for example, twenty (20) minutes to thirty (30) minutes.

The formula 3 may be cooled to a second temperature at step 700. The second temperature may be between, for example, fifty (50) degrees Celsius and fifty-five (55) degrees Celsius. A third moisturizer 40, an absorbant 42 and/or a UV blocker 44 may be added to the formula 3 at step 800. The third moisturizer 40 may be, for example, mineral oil. The absorbant 42 may be, for example, corn starch or Zea Mays starch. The UV blocker 44 may be, for example, zinc oxide and/or the like. The formula 3 may be mixed for a duration of time between, for example, twenty-five (25) minutes and thirty (30) minutes. As a result, the formula 3 may be homogeneous and/or the compounding 1 of the formula 3 may be completed.

The formula 3, the system and/or the method may protect the area of the skin of the user against dermatitis, sunlight and/or insects. The formula 3 and/or the system may be applied to an area of skin of a user to prevent the dermatitis, the sunlight and/or the insect from affecting the area. The formula and/or the system may have UV absorbers 14, 16 and/or a UV blocker 44 to prevent effects of the sunlight on the area of the skin. Additionally, the UV blocker 44 may protect the area of the skin from dermatitis and/or may prevent an insect from biting the area of the skin of the user.

It should be understood that various changes and modifications to the presently preferred embodiments described herein will be apparent to those skilled in the art. Such changes and modifications may be made without departing from the spirit and scope of the present invention and without diminishing its attendant advantages. It is, therefore, intended that such changes and modifications be covered by the appended claims.

We claim:

1. A formula for providing protection of an area of skin of a user from dermatitis or an insect wherein the formula is applied to the area of the skin of the user, the formula comprising:
an ultraviolet light blocker wherein the ultraviolet light blocker prevents the area of the skin from being affected by dermatitis or an insect;
a first ultraviolet light absorber;
a second ultraviolet light absorber;
a plurality of moisturizing agents; and
an absorbant.

2. The formula of claim 1 wherein the ultraviolet light blocker is zinc oxide.

3. The formula of claim 1 wherein the first ultraviolet light absorber is ethylhexyl methoxycinnamate or benzophenone-3.

4. The formula of claim 1 wherein the second ultraviolet light absorber is ethylhexyl methoxycinnamate or benzophenone-3.

5. The formula of claim 1 further comprising:
a wax.

6. The formula of claim 1 wherein the absorbant is corn starch.

7. The formula of claim 1 further comprising:
an anti-microbial agent.

8. The formula of claim 1 further comprising:
an anti-fungal agent.

9. A formula for providing protection of an area of skin of a user from dermatitis or an insect wherein the formula is applied to the area of the skin of the user, the formula comprising:
an ultraviolet light blocker to protect the area of the skin from effects of the dermatitis and the insect;
a first ultraviolet light absorber to prevent the sunlight from affecting the skin of the user; and
petrolatum.

10. The formula of claim 9 wherein the ultraviolet light blocker is zinc oxide.

11. The formula of claim 9 wherein the first ultraviolet light absorber is ethylhexyl methoxycinnamate or benzophenone-3.

12. The formula of claim 9 further comprising:
an anti-microbial agent.

13. The formula of claim 9 further comprising:
a second ultraviolet light absorber.

14. A method for providing protection of an area of skin of a user from dermatitis or an insect, the method comprising the steps of:
providing a formula to block the dermatitis or the insect from affecting the area of the skin of the user wherein the formula has a zinc oxide, a first ultraviolet absorber, a petrolatum and an anti-microbial agent therein and further wherein the first ultraviolet absorber is ethylhexyl methoxycinnamate or benzophenone-3; and
applying the formula to the area of the skin prior to exposing the area of the skin to the dermatitis or the insects.

15. The method of claim 14 further comprising the step of:
adding a moisturizing agent to the formula.

16. The method of claim 14 further comprising the step of:
adding a second ultraviolet absorber to the formula.

17. The method of claim 14 further comprising the step of:
adding an absorbant to the formula.

18. The method of claim 14 further comprising the step of:
adding a wax to the formula.

19. The method of claim 14 further comprising the step of:
homogenizing the formula.

20. The method of claim 14 wherein the anti-microbial agent is methylparaben or propylparaben.
